(19) United States
(12) Patent Application Publication
(10) Pub. No.: US 2022/0050078 A1
(43) Pub. Date: Feb. 17, 2022

(54) GEL, MARKER, AND KIT FOR PROTEIN ELECTROPHORESIS, AND APPLICATION OF GEL

(71) Applicant: Innovation Academy for Precision Measurement Science and Technology, Chinese Academy of Sciences, Wuhan (CN)

(72) Inventors: Liqun HUANG, Wuhan (CN); Ling JIANG, Wuhan (CN); Conggang LI, Wuhan (CN)

(21) Appl. No.: 17/513,935

(22) Filed: Oct. 29, 2021

Related U.S. Application Data
(63) Continuation-in-part of application No. PCT/CN2020/087126, filed on Apr. 27, 2020.

(30) Foreign Application Priority Data
Apr. 29, 2019 (CN) 201910354425.5
Jun. 29, 2021 (CN) 202110723864.6

(51) Int. Cl. G01N 27/447; G01N 33/68

(52) U.S. Cl. CPC G01N 27/44747; G01N 27/44778; G01N 27/44739; G01N 33/6803

(57) ABSTRACT

A gel for protein electrophoresis includes a separating gel and a stacking gel disposed on the separating gel. The separating gel is a polyacrylamide gel including a surfactant, and is alkaline. The surfactant of the separating gel includes 0.025-0.1% (m/v) of sodium lauroyl sarcosinate. The ratio of the molar concentration of the surfactant to the mass concentration of a loading protein is between 0.04 mmol/g and 11.56 mmol/g.

### CROSS-REFERENCE TO RELAYED APPLICATIONS

This application is a continuation-in-part of International Patent Application No. PCT/CN2020/087126 with an international filing date of Apr. 27, 2020, designating the United States, now pending, and further claims foreign priority benefits to Chinese Patent Application No. 201910354425.5 filed Apr. 29, 2019, and to Chinese Patent Application No. 202110723864.6 filed Jun. 29, 2021. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of bioengineering, and more particularly, to a gel, a Marker, and a kit for protein electrophoresis, and an application of the gel.

Currently, the study on protein morphology and structure requires separating the protein, and then extracting a single type of proteins. One common method for protein separation is gel electrophoresis, for example, sodium dodecyl sulfate polyacrylamide gel electrophoresis (SDS-PAGE) and Native-PAGE. Gel electrophoresis is a laboratory method used to separate mixtures of proteins according to molecular size. In gel electrophoresis, the molecules to be separated are pushed by an electrical field through a gel that comprises small pores. The molecules travel through the pores in the gel at a speed that is inversely related to their molecular weights.

Sodium dodecyl sulfate (SDS) is a strong anionic detergent and can bind to a protein to denature the globular structure thereof, so that the molecular mass of a protein is related to the quantity of bound electric charges thereof. Therefore, the migration rate of a protein in the electrophoresis process depends only on the molecular mass of the protein, and a protein Marker can be used to indicate the molecular mass of a target protein. Since SDS-PAGE destroys the native conformation of a protein and is often used in the study of denatured proteins, nearly no modified state or complex state of the protein is observed in SDS-PAGE.

Native-PAGE is a non-denaturing gel electrophoresis without SDS. During the electrophoresis process, the protein remains intact. Because the migration rate of protein in Native-PAGE is determined by the molecular mass, protein shape and quantity of electric charges, there is no standard protein Marker in the Native-PAGE, which brings troubles for the analysis of protein molecular mass, polymerization state and complex state; in addition, the difference of the quantity of electric charges between acidic protein and basic protein brings inconvenience to the experimental operation.

### SUMMARY

The disclosure provides a gel, a Marker, and a kit for protein electrophoresis, and an application of the gel. The gel of the disclosure does not destroy the secondary structure of a protein and can be used for calibration and semi-quantitative analysis on a target protein through electrophoresis, with simple operation and low costs, solving the technical problem in the related art that it is difficult to perform the protein mass analysis through gel electrophoresis under a weak protein interaction.

The disclosure provides a gel for protein electrophoresis comprising a separating gel; the separating gel is a polyacrylamide gel comprising a surfactant, and is alkaline; the surfactant of the separating gel comprises 0.025-0.1% (m/v) of sodium lauroyl sarcosinate (SAR); a ratio of a molar concentration of the surfactant to a mass concentration of a loading protein is between 0.04 mmol/g and 11.56 mmol/g, preferably between 1.16 mmol/g and 9.90 mmol/g.

In a class of this embodiment, a stacking gel of the gel for protein electrophoresis is a polyacrylamide gel comprising a surfactant, and is weakly acidic. Preferably, the stacking gel comprises polyacrylamide with a mass volume fraction of 3.5-5%, and the surfactant of the stacking gel comprises 0.025-0.1% (m/v) of SAR.

In another aspect of the disclosure, a buffer for protein electrophoresis comprising a surfactant is provided. The surfactant comprises 0.025-0.1% (m/v) of SAR, and the surfactant concentration and the concentration of loading protein has the following relationship:

The ratio of the molar concentration of the surfactant to the mass concentration of the loading protein is between 0.04 mmol/g and 11.56 mmol/g.

In a class of this embodiment, the buffer for protein electrophoresis is a loading buffer, a running buffer, or a buffer for dissolving a protein Marker.

In a class of this embodiment, the buffer for protein electrophoresis is a loading buffer, and the mass concentration of the loading protein is not more than 40.32 mg/mL.

In a class of this embodiment, the mass concentration of the loading protein for the buffer for protein electrophoresis is 0.59-2.94 mg/mL.

In still another aspect of the disclosure, a Marker for protein electrophoresis is provided, which comprises 4 to 20 kinds of stabilized monomer proteins with a molecular mass distribution range of 5 kDa to 100 MDa, and a total protein concentration of 0.005 mmol/L to 0.5 mmol/L. The stabilized monomer proteins refer to proteins used as a Marker, and no polymer is formed in the proteins themselves or between the proteins; a dissolution buffer comprises SAR with the same concentration as the stacking gel.

In a class of this embodiment, the Marker for protein electrophoresis comprises 4 to 20 kinds of stabilized monomer proteins.

In still another aspect of the disclosure, applications of the gel for protein electrophoresis are provided. Specifically, the disclosure provides a method for separation and determination of polymerization states of a protein, observation of modification states of a protein, observation of protein oligomer, analysis of protein purity, determination of protein molecular mass, protein identification by Western Blotting, detection of aggregation states of intracellular and extracellular membrane proteins, or identification of protein complexes separated by two-dimensional 05SAR/SDS-PAGE, the method comprising applying the gel for protein electrophoresis.

In a class of this embodiment, when the gel for protein electrophoresis is applied to separation and determination of polymerization states of proteins, observation of protein complexes, analysis of protein purity, determination of protein molecular mass, or detection of aggregation states of intracellular and extracellular membrane proteins, the ratio of a molar concentration of the surfactant to the mass concentration of the loading protein is between 0.04 mmol/g and 11.56 mmol/g.

In a class of this embodiment, when the gel for protein electrophoresis is applied to observation of modification states of a protein, the ratio of a molar concentration of the surfactant to the mass concentration of the loading protein is between 0.17 mmol/g and 2.31 mmol/g.

In a class of this embodiment, when the gel for protein electrophoresis is applied to protein identification by Western Blotting, or identification of protein complexes separated by two-dimensional 05SAR/SDS-PAGE, the ratio of a molar concentration of the surfactant to the mass concentration of the loading protein is between 0.04 mmol/g and 5.31 mmol/g.

In still another aspect of the disclosure, a kit for protein gel electrophoresis is provided. The kit comprises a stock solution of the stacking gel and a stock solution of the separating gel of the gel for protein electrophoresis provided by the disclosure.

In a class of this embodiment, the kit for protein gel electrophoresis further comprises a Marker for protein electrophoresis provided by the disclosure.

In a class of this embodiment, the stock solution of the stacking gel comprises a buffer comprising SAR and acrylamide at a formulation concentration; and the stock solution of the separating gel comprises a buffer comprising SAR and acrylamide at a formulation concentration, ammonium persulfate solution at a concentration of not less than 0.01% w/v, and tetramethylethylenediamine (TEMED) solution.

The following advantages are associated with the gel for protein electrophoresis of the disclosure:

Firstly, the gel of the disclosure has the characteristics of traditional SDS-PAGE and Native-PAGE that can separate proteins in different states and the operation is simple and convenient, and overcomes the shortcomings that the SDS-PAGE cannot be used directly for analysis of protein modification in a physiological state and the Native-PAGE cannot be used for calibration with protein Marker and simultaneous analysis of acidic proteins and basic proteins; and the gel of the disclosure can be used to calibrate and semi-quantitatively analyze the target protein of electrophoresis without destroying the secondary structure of the protein.

Secondly, the experimental results have shown that this method is universal in applicability, simple in operation and low in cost.

Thirdly, the disclosure can be widely used in basic and applied research in the fields of molecular dynamics, enzymology, medicine, etc., to provide convenience for the scientific analysis of proteins.

### BRIEF DESCRIPTION OF THE DRAWINGS

FIGS. 1A-1B are gel electrophoresis patterns of 05SAR-PAGE and 05SAR-PAGE of Example 1; FIG. 1A is the result of protein Marker and CpxA^{CA} protein electrophoresis in 05SAR-PAGE; FIG. 1B is the result of protein Marker and PDI electrophoresis in the 05SAR-PAGE patterns;

FIG. 2 is a ¹-¹⁵N HSQC spectrum of PhoB_{N} of Example 2 and the ¹-¹⁵N HSQC spectrum of PhoB_{N} with 0.01% (w/v) SAR added;

FIG. 3 is a ¹-¹⁵N HSQC spectrum of PhoB_{N} of Example 2 and the ¹-¹⁵N HSQC spectrum of PhoB_{N} with 0.1% (w/v) SAR added;

FIGS. 4A-4C are ¹-¹⁵N HSQC spectra of PhoB_{N} collected in Example 3 using 0.05% (w/v) SAR to titrate different concentrations of PhoB_{N}; FIG. 4A is a ¹-¹⁵N HSQC spectrum after adding 0.05% (w/v) SAR to 0.01 mmol/L PhoB_{N}; FIG. 4B is a ¹H-¹⁵N HSQC spectrum after adding 0.05% (w/v) SAR to 0.10 mmol/L PhoB_{N}; FIG. 4C is a ¹-¹⁵N HSQC spectrum after adding 0.05% (w/v) SAR to 1.00 mmol/L PhoB_{N};

FIG. 5 is a 05SAR-PAGE result of Cyt c with different concentrations (0.2 mmol/L, 0.4 mmol/L, 0.8 mmol/L, 1.6 mmol/L, 3.2 mmol/L) in Example 4;

FIG. 6 is a relationship diagram between the relative molecular mass and the number of bound SARs in Example 5;

FIGS. 7A-7B are relationship diagram between the molecular mass of the protein and the migration rate of the protein in the 05SAR-PAGE of Example 5, FIG. 7A is the 05SAR-PAGE result of the protein Marker; FIG. 7B is a relative relationship between the logarithm of the relative molecular mass of the protein Marker and the relative migration rate of the protein in the 05SAR-PAGE result;

FIG. 8 is the result of the oligomerization states of 0.5 mmol/L Cyt c identified by 05SAR-PAGE in Example 6;

FIGS. 9A-9B are electrophoresis patterns of PhoR_{CP} in Example 7; FIG. 9A is a SDS-PAGE pattern of 0.3 mmol/L PhoR_{CP}; FIG. 9B is a 05SAR-PAGE pattern of 0.3 mmol/L PhoR_{CP};

FIGS. 10A-10B are gel electrophoresis patterns of PhoB_{N} in Example 7; FIG. 10A is a SDS-PAGE pattern of 0.1 mmol/L PhoB_{N}; FIG. 10B is a 05SAR-PAGE pattern of 0.04 mmol/L-0.2 mmol/L PhoB_{N};

FIGS. 11A-11B are gel electrophoresis patterns of CpxA in Example 8; FIG. 11A is a 05SAR-PAGE pattern of CpxA; and FIG. 11B is a SDS-PAGE pattern of CpxA;

FIGS. 12A-12B are results of modified states of a protein identified by 05SAR-PAGE in Example 9; FIG. 12A is a result of 0.3 mmol/L PhoB_{N} at phosphorylated and non-phosphorylated states identified by 05SAR-PAGE; FIG. 12B is a result of unmethylated Cyt c and methylated Cyt c identified by SDS-PAGE and 05SAR-PAGE;

FIG. 13 is the relative quantification result of CpxA in E. coli cell lysates of different concentrations (different lanes) by 05SAR-PAGE in Example 10 combined with Western blotting.

FIGS. 14A-14B are patterns of 05SAR-PAGE and Western Blotting to analyze the dimerization state of the soluble protein PhoB_{N} and membrane protein CpxA in the cell lysate in Example 10; FIG. 14A is patterns of 05SAR-PAGE and Western Blotting to analyze the dimerization state of the soluble protein PhoB_{N} in the cell lysate; FIG. 14B is patterns of 05SAR-PAGE and Western Blotting to analyze the dimerization state of the membrane protein CpxA in the cell lysate.

FIG. 15 is a schematic diagram for the construction of two-dimensional 05SAR/SDS-PAGE and mass spectrometry in Example 11.

FIG. 16 is a result diagram of CpxA complex identified by two-dimensional 05SAR/SDS-PAGE in Example 11.

### DETAILED DESCRIPTION

To further illustrate, embodiments detailing a gel, a Marker, and a kit for protein electrophoresis, and an application of the gel are described below. It should be noted that the following embodiments are intended to describe and not to limit the disclosure.

The gel for protein electrophoresis provided by the disclosure comprises a stacking gel and a separating gel disposed below the stacking gel.

The separating gel is a polyacrylamide gel comprising a surfactant, preferably comprising polyacrylamide with a mass fraction of 6% to 15%. The surfactant of the separating gel comprises 0.025-0.1% (m/v) of SAR. The pH value of the separating gel is basic, between 8.0 and 9.5, preferably 8.8. The concentration of the surfactant and the concentration of the loading protein have the following relationship:

The ratio of the molar concentration of the surfactant to the mass concentration of the loading protein is between 0.04 mmol/g and 11.56 mmol/g, preferably between 1.16 mmol/g and 9.90 mmol/g.

Experiments have shown that the surface of protein is bound to SAR. Since the volume of the protein is much larger than that of SAR, the ratio of the molar concentration of SAR to the mass concentration of the loading protein determines the performance of SAR gel electrophoresis within a certain range. Experiments show that, when the ratio of the molar concentration of the surfactant to the mass concentration of the loading protein is more than 11.56 mmol/g, protein denaturation occurs and the native conformation cannot be maintained; when the ratio of the molar concentration of the surfactant to the mass concentration of the loading protein is lower than 0.04 mmol/g, the resolution of gel electrophoresis may decrease due to insufficient SAR binding on the protein surface, thus it is unable to distinguish proteins with similar molecular masses. According to the experimental results of protein denaturation and gel imaging resolution, when the ratio of the molar concentration of the surfactant to the mass concentration of the loading protein is between 0.04 mmol/g and 11.56 mmol/g, the protein electrophoresis provided by the disclosure can have better resolution and maintain the native conformation of proteins. When the ratio of the molar concentration of the surfactant to the mass concentration of the loading protein is between 1.66 mmol/g and 9.90 mmol/g, the molecular mass of the protein has a good linear relationship with the average migration rate, which can more accurately mark the molecular mass of the protein and has a good resolution.

The stacking gel is a polyacrylamide gel comprising a surfactant, preferably comprising polyacrylamide with a mass volume fraction of 3.5-5%, with a weakly acidic pH between 6.0 and 7.0, preferably 6.8. The surfactant of the stacking gel comprises 0.025-0.1% (m/v) of SAR.

Buffers for gel electrophoresis provided by the disclosure, such as a loading buffer, a running buffer, a buffer for dissolving a protein Marker, contain a surfactant, and the surfactant comprises 0.025-0.1% (m/v) of SAR, and the surfactant concentration and the concentration of loading protein has the following relationship:

The ratio of the molar concentration of the surfactant to the mass concentration of the loading protein is between 0.04 mmol/g and 11.56 mmol/g.

Preferably, the mass concentration of the loading protein is not more than 40.32 mg/mL, preferably 0.59-2.94 mg/mL. When the concentration of the loading protein is more than 40.32 mg/mL, the band tailing is serious and the protein cannot be separated clearly.

The disclosure provides a Marker for protein electrophoresis, which comprises 4 to 20 kinds of stabilized monomer proteins, with a molecular mass distribution range of 5 kDa to 100 MDa (distributed evenly as much as possible), and a total protein concentration of 0.005 mmol/L to 0.5 mmol/L; the stabilized monomer proteins refer to proteins used as a Marker, and no polymer is formed in the proteins themselves or between the proteins; a dissolution buffer comprises SAR with the same concentration as the stacking gel. Since the protein electrophoresis gel provided by the disclosure is often used for the observation of multimeric proteins, the molecular mass range of electrophoresis Marker is correspondingly expanded, and a stabilized monomer protein with a wider distribution range needs to be selected.

In the running buffer of SDS gel electrophoresis, proteins have a precise elliptical or rod-shaped structure due to the strong denaturation of SDS. The size of short axis of the rod is basically constant, regardless of the type of protein; while the size of long axis of the rod changes and is directly proportional to the molecular mass of the protein. In addition, SDS is an anionic surfactant, so the protein surface is negatively charged, and a long rod shape whose length is proportional to the molecular mass of the protein is formed, which eliminates the influence on the migration rate of different electrophoresis caused by the difference in the shape of natural protein molecules. The migration rate of electrophoresis of charged molecules depends on three factors, namely quantity of electric charges, molecular mass, and shape of molecules. As described above, after SDS binds to a protein, the migration rate is only determined by the molecular mass of the protein. Therefore, in SDS-gel electrophoresis, the molecular mass of the unknown protein can be determined by comparing the migration rate of the protein of unknown molecular mass with that of known molecular mass. However, due to the strong interaction of SDS with proteins, the structure of most protein is destroyed. Thus, it is almost impossible to conduct gel electrophoresis while maintaining weak interactions between proteins. For example, the proteins in an oligomerized, complex, modified and polymerized state are hardly identified because their structures are disrupted by SDS.

Native-PAGE is a gentle non-denaturing gel electrophoresis without adding a surfactant. During the electrophoresis process, the protein can remain intact. Because the migration speed of the protein in Native-PAGE is affected by the molecular mass, protein shape, and quantity of electric charge, it is impossible to calibrate with a unified protein Marker for Native-PAGE, which will cause troubles for analyzing the molecular mass, polymerization states and complex states of proteins. In addition, due to the difference in the quantity of electric charges between acidic protein and basic protein, the proteins cannot be separated in one step, which brings inconvenience to the experimental operation. In other words, Native-PAGE can be used to distinguish different proteins, but hardly be used to determine the molecular mass of proteins.

During SDS gel electrophoresis, almost all proteins with weak interactions cannot maintain weak interactions due to changes in protein morphology. Therefore, it is impossible to identify weakly interacting proteins by SDS gel electrophoresis, However, for non-denaturing gel electrophoresis, the influence of factors other than molecular mass on the electrophoresis results cannot be excluded, especially the influence caused by electric charges, therefore, it is almost impossible to identify weak interactions and the molecular mass of proteins simultaneously by non-denaturing gel electrophoresis.

In the disclosure, SAR is used to replace SDS as a surfactant for protein gel electrophoresis, preferably the surfactant for active protein gel electrophoresis is used, such as weak protein interaction gel electrophoresis, in-situ protein gel electrophoresis, etc. While appropriately controlling the molar concentration of the loading protein and the amount of the surfactant and under the conditions of maintaining the native conformation of protein and maintaining the weak interaction of protein without destroying the protein activity, the protein is negatively charged and the effect of the protein's natural electric quantity on the results of gel electrophoresis is shielded, to achieve the identification of the weak interactions of proteins. The ratio of the molar concentration of the surfactant to the mass concentration of the loading protein is between 0.04 mmol/g and 11.56 mmol/g.

As an anionic surfactant, SAR is also bound to the protein surface. The titration experiment of Example 5 showed (FIG. 6) that, when the ratio of the molar concentration of SAR to the mass concentration of the loading protein is in the range of 1.66 mmol/g to 9.90 mmol/g, the molecular mass of the protein and the amount of bound SAR exhibited a good linear relationship. Therefore, at a suitable pH value, the native conformation of the protein is not destroyed, and the SAR is in an ionized state, so that the protein surface is negative charged e that is linearly positively related to the molecular mass of the protein while maintain the active state of the protein comprising weak interaction, thereby shielding the effect of the quantity of charges on the result of gel electrophoresis, making the migration speed of the protein in gel electrophoresis only related to the size and shape of the protein.

Since the difference in molecular mass of different proteins has a much greater impact on the migration speed of the gel electrophoresis than their shapes, the position of the protein band basically identifies the protein molecular mass when analyzing different proteins. When molecular masses of proteins are very similar, SAR-protein gel electrophoresis may be able to distinguish proteins of different shapes, for example, chemically modified proteins or proteins in different conformations. Therefore, the SAR-protein gel electrophoresis provided by the disclosure has a higher resolution than Native-protein gel electrophoresis.

More importantly, the disclosure provides a new method for detecting weak protein interactions, which is different from the chromatographic analysis, nuclear magnetic resonance spectroscopy and analytical centrifugation commonly used in the detection of weak protein interactions. The SAR-protein gel electrophoresis provided in the disclosure is used for detecting weak protein interactions, which overcomes the disadvantages that Native gel electrophoresis cannot be used for calibration of protein molecular mass through the protein Marker and cannot be used for separating the acidic and basic proteins in one step. SAR-gel electrophoresis can be used for detecting the oligomerization states of proteins through the protein Marker, and analyzing the acidic proteins and basic proteins in one step, and identifying the modification states of proteins and the modification states of the polymerized proteins.

Further, the SAR-protein gel electrophoresis provided by the disclosure can be combined with Western Blotting to achieve in-situ detection of weak protein interactions, for example, detecting the polymerization states of intracellular proteins; in addition, it can detect the aggregation states of membrane proteins, and can be combined with 05SAR-PAGE/SDS-PAGE to separate protein complexes through two-dimensional electrophoresis. Based on the molecular mass of the target protein and the concentration of polyacrylamide in SAR-protein gel electrophoresis, the best separation effect can be achieved. The SAR-protein gel electrophoresis has a wide application range, a low cost, simple operation and data analysis, and more intuitive results.

For the SAR-protein gel electrophoresis provided by the disclosure, the control of the SAR concentration and the protein concentration is the key point to perform SAR-gel electrophoresis while maintaining the active state of the protein. Specifically, when the molar concentration of SAR and the mass concentration of the loading protein are between 0.04 mmol/g and 11.56 mmol/g, the state of weak protein interactions can be maintained without destroying the protein structure. If the ratio is too large, the native conformation of the protein will be destroyed. If the ratio is too small, there is no obvious linear relationship between the quantity of electric charges on the surface of the protein and the molecular mass of the protein, then the resolution of SAR-protein gel electrophoresis will deteriorate, and more importantly, it is impossible to accurately identify the molecular mass of the protein. Both of the above two situations will cause failure to SAR-gel electrophoresis.

Furthermore, according to the different types and natures of the weak interaction of the target proteins, different SAR concentrations, different ratios of SAR concentration to the concentration of the loading protein, and different polyacrylamide concentrations present different identification effects.

When the gel of the disclosure is used for direct separation and determination of polymerization states of proteins, the suitable ratio of the molar concentration of the surfactant to the mass concentration of the loading protein is 0.04-11.56 mmol/g, preferably the pH value of the separating gel is 8.8, and the pH value of the stacking gel is 6.8.

When the gel of the disclosure is used for observation of modification states of protein, since the molecular mass of the modified protein is very close to that of the non-modified protein, the gel electrophoresis should have higher resolution and better maintenance of the native conformation of the protein. Therefore, the ratio of the molar concentration of the surfactant to the mass concentration of loading protein is between 0.17 mmol/g and 2.70 mmol/g.

When the gel of the disclosure is used for observation of a protein complex, the suitable ratio of the molar concentration of the surfactant to the mass concentration of the loading protein is 0.04-11.56 mmol/g.

When the gel of the disclosure is used for analysis of protein purity and determination of protein molecular mass, it is preferable to analyze the monomer target protein, and the suitable ratio of the molar concentration of the surfactant to the mass concentration of the loading protein is 0.04-11.56 mmol/g.

When the disclosure replaces SDS-PAGE for Western Blotting to observe the polymerization states of intracellular proteins and the polymerization states of membrane proteins, the suitable ratio of the molar concentration of SAR to the mass concentration of the loading protein is 0.04-5.31 mmol/g. Because the staining method of Western Blotting is more sensitive than the above-mentioned gel, the protein can be detected when the concentration is low.

When the disclosure is used for identification of protein complexes separated by two-dimensional 05SAR/SDS-PAGE, the suitable ratio of the molar concentration of SAR to the mass concentration of the loading protein is 0.04-5.31 mmol/g. Since the lower the concentration of polyacrylamide, the larger the pore size of the gel, and the larger the molecular mass of the separated protein. Therefore, different concentrations of polyacrylamide and the protein Marker suitable for the molecular mass of the target protein can be chosen according to the molecular mass of the proteins to be separated.

When the disclosure is used for detection of the aggregation states of intracellular and extracellular membrane proteins, the suitable ratio of the molar concentration of the surfactant to the mass concentration of the loading protein is 0.04-11.56 mmol/g.

The kit for protein gel electrophoresis provided by the disclosure comprises a stock solution of the stacking gel and a stock solution of the separating gel, preferably, further comprises the electrophoresis Marker provided by the disclosure.

The stock solution of the stacking gel comprises a buffer comprising SAR and acrylamide at a formulation concentration; and the stock solution of the separating gel comprises a buffer comprising SAR and acrylamide at a formulation concentration, ammonium persulfate solution, and TEMED solution; the concentration of ammonium persulfate solution is generally not less than 0.01% w/v.

The protein separation gel used in the following examples comprises a separating gel and a stacking gel:

The length and thickness of the separating gel are the same as that of the stacking gel.

The height of the separating gel: the height of the stacking gel=2:1 to 8:1.

The preparation method is as follows:

1. Preparation of Gel Plate

A. Preparation of separating gel

a. Prepare the separating gel according to the amount in the separating gel formula table. In a clean small beaker, add distilled water and other separating gel formulation ingredients except tetramethylethylenediamine (TEMED). Mix gently, and finally add TEMED, mix gently again;

b. Mix the above mixture with a 1 mL pipette and add it to the gap between the long and short gel-making plates. The separating gel is 53-63 mm high. Use a 1 mL syringe to take a little distilled water and fill the water seal along the long glass plate, 15-30 min later, absorb the excess water using a filter paper.

B. Preparation of stacking gel

a. Prepare the staking gel according to the amount in the stacking gel formula table. In a clean small beaker, add distilled water and other separating gel formulation ingredients except tetramethylethylenediamine (TEMED). Mix gently, and finally add TEMED, mix gently again;

b. Use a 1 mL pipette to mix the above mixture and add it to the stacking gel, quickly insert the gel combs, and place it for 20 to 60 min; when the staking gel is completely solidified, take out the gel comb and place it in a refrigerator at 4° C. for later use;

2. Preparation of Loading Buffer

A. Prepare corresponding stock solution according to the loading buffer formula table;

B. Take the stock solution at a formulation amount, add deionized water to dissolve and dilute to volume to 5 mL;

C. Aliquot and store at room temperature.

3. Preparation of Running Buffer:

A. Prepare corresponding stock solution according to the running buffer formula table;

B. Add deionized water to dissolve and dilute to volume to 500 mL;

4. Preparation of Protein Marker

The protein Marker comprises 4 to 8 molecular mass gradients, and the protein molecular mass ranges from 5 kDa to 300 kDa. The protein Marker with a concentration of 0.05-0.5 mmol/L is dissolved in the loading buffer. The protein Marker used in the embodiment is a commercially available protein Marker added with SAR. The commercially available protein Marker is purchased from ThermoFisher (MA, USA), with a molecular mass ranging from 10 kDa to 180 kDa.

5. Preparation of Protein Samples

Mix the protein sample to be analyzed with the loading buffer at a volume ratio of 1:1, to obtain the final protein sample. The concentration of protein sample is controlled at 0.05-0.5 mM.

### Example 1

Separation of CpxA^{CA} and PDI proteins by 05SAR-PAGE

1. Preparation of a gel plate:

TABLE 1
Formula for preparation of separating gel for
Tris-Glycine 6% 05SAR-PAGE

| | Volume (mL) of each component in | | | | | | | |
|---|---|---|---|---|---|---|---|---|
| Com- | different volumes (mL) of separating gel | | | | | | | |
| ponent | 5 | 10 | 15 | 20 | 25 | 30 | 40 | 50 |
| dd H₂O | 2.6 | 5.3 | 7.9 | 10.6 | 13.2 | 15.9 | 21.2 | 26.5 |
| 30% | 1 | 2 | 3 | 4 | 5 | 6 | 8 | 10 |
| Acryl- | | | | | | | | |
| amide | | | | | | | | |
| solution | | | | | | | | |
| 5% SAR | 0.05 | 0.1 | 0.15 | 0.2 | 0.25 | 0.3 | 0.4 | 0.5 |
| 1.5 mol/L | 1.3 | 2.5 | 3.8 | 5 | 6.3 | 7.5 | 10 | 12.5 |
| Tris | | | | | | | | |
| (pH 8.8) | | | | | | | | |
| 10% | 0.05 | 0.1 | 0.15 | 0.2 | 0.25 | 0.3 | 0.4 | 0.5 |
| Ammon- | | | | | | | | |
| ium | | | | | | | | |
| persulfate | | | | | | | | |
| TEMED | 0 | 0.01 | 0.012 | 0.016 | 0.02 | 0.024 | 0.03 | 0.04 |

TABLE 2
Formula for preparation of separating gel for
Tris-Glycine 8% 05SAR-PAGE

| | Volume (mL) of each component in | | | | | | | |
|---|---|---|---|---|---|---|---|---|
| | different volumes (mL) of separating gel | | | | | | | |
| Component | 5 | 10 | 15 | 20 | 25 | 30 | 40 | 50 |
| dd H₂O | 2.3 | 4.6 | 6.9 | 9.3 | 11.5 | 13.9 | 18.5 | 23.2 |
| 30% Acrylamide | 1.3 | 2.7 | 4 | 5.3 | 6.7 | 8 | 10.7 | 13.3 |
| solution | | | | | | | | |
| 5% SAR | 1.3 | 2.5 | 3.8 | 5 | 6.3 | 7.5 | 10 | 12.5 |
| 1.5 mol/L Tris | 0.05 | 0.1 | 0.15 | 0.2 | 0.25 | 0.3 | 0.4 | 0.5 |
| (pH 8.8) | | | | | | | | |
| 10% Ammonium | 0.05 | 0.1 | 0.15 | 0.2 | 0.25 | 0.3 | 0.4 | 0.5 |
| persulfate | | | | | | | | |
| TEMED | 0.003 | 0.006 | 0.009 | 0.012 | 0.015 | 0.018 | 0.024 | 0.03 |

TABLE 3
Formula for preparation of separating gel for
Tris-Glycine 10% 05SAR-PAGE

| | Volume (mL) of each component in | | | | | | | |
|---|---|---|---|---|---|---|---|---|
| | different volumes (mL) of separating gel | | | | | | | |
| Component | 5 | 10 | 15 | 20 | 25 | 30 | 40 | 50 |
| dd H₂O | 1.9 | 4 | 5.9 | 7.9 | 9.9 | 11.9 | 15.9 | 19.8 |
| 30% Acrylamide | 1.7 | 3.3 | 5 | 6.7 | 8.3 | 10 | 13.3 | 16.7 |
| solution | | | | | | | | |
| 5% SAR | 1.3 | 2.5 | 3.8 | 5 | 6.3 | 7.5 | 10 | 12.5 |
| 1.5 mol/L Tris | 0.05 | 0.1 | 0.15 | 0.2 | 0.25 | 0.3 | 0.4 | 0.5 |
| (pH 8.8) | | | | | | | | |
| 10% Ammonium | 0.05 | 0.1 | 0.15 | 0.2 | 0.25 | 0.3 | 0.4 | 0.5 |
| persulfate | | | | | | | | |
| TEMED | 0.002 | 0.004 | 0.006 | 0.008 | 0.01 | 0.012 | 0.016 | 0.02 |

TABLE 4
Formula for preparation of separating gel for
Tris-Glycine 12% 05SAR-PAGE

| | Volume (mL) of each component in | | | | | | | |
|---|---|---|---|---|---|---|---|---|
| | different volumes (mL) of separating gel | | | | | | | |
| Component | 5 | 10 | 15 | 20 | 25 | 30 | 40 | 50 |
| dd H₂O | 1.6 | 3.3 | 4.9 | 6.6 | 8.2 | 9.9 | 13.2 | 16.5 |
| 30% Acrylamide | 2 | 4 | 6 | 8 | 10 | 12 | 16 | 20 |
| solution | | | | | | | | |
| 5% SAR | 1.3 | 2.5 | 3.8 | 5 | 6.3 | 7.5 | 10 | 12.5 |
| 1.5 mol/L Tris | 0.05 | 0.1 | 0.15 | 0.2 | 0.25 | 0.3 | 0.4 | 0.5 |
| (pH 8.8) | | | | | | | | |
| 10% Ammonium | 0.05 | 0.1 | 0.15 | 0.2 | 0.25 | 0.3 | 0.4 | 0.5 |
| persulfate | | | | | | | | |
| TEMED | 0.002 | 0.004 | 0.006 | 0.008 | 0.01 | 0.012 | 0.016 | 0.02 |

TABLE 5
Formula for preparation of separating gel for
Tris-Glycine 15% 05SAR-PAGE

| | Volume (mL) of each component in | | | | | | | |
|---|---|---|---|---|---|---|---|---|
| | different volumes (mL) of separating gel | | | | | | | |
| Component | 5 | 10 | 15 | 20 | 25 | 30 | 40 | 50 |
| dd H₂O | 1.1 | 2.3 | 3.4 | 4.6 | 5.7 | 6.9 | 9.2 | 11.5 |
| 30% Acrylamide | 2.5 | 5 | 7.5 | 10 | 12.5 | 15 | 20 | 25 |
| solution | | | | | | | | |
| 5% SAR | 1.3 | 2.5 | 3.8 | 5 | 6.3 | 7.5 | 10 | 12.5 |
| 1.5 mol/L Tris | 0.05 | 0.1 | 0.15 | 0.2 | 0.25 | 0.3 | 0.4 | 0.5 |
| (pH 8.8) | | | | | | | | |
| 10% Ammonium | 0.05 | 0.1 | 0.15 | 0.2 | 0.25 | 0.3 | 0.4 | 0.5 |
| persulfate | | | | | | | | |
| TEMED | 0.002 | 0.004 | 0.006 | 0.008 | 0.01 | 0.012 | 0.016 | 0.02 |

TABLE 6
Formula for preparation of stacking gel for Tris-Glycine 05SAR-PAGE

| | Volume (mL) of each component in | | | | | |
|---|---|---|---|---|---|---|
| | different volumes (mL) of stacking gel | | | | | |
| Component | 2 | 3 | 4 | 6 | 8 | 10 |
| dd H₂O | 1.12 | 1.68 | 2.24 | 3.36 | 4.48 | 5.6 |
| 30% Acrylamide | 0.34 | 0.51 | 0.68 | 1.02 | 1.36 | 1.7 |
| solution | | | | | | |
| 5% SAR | 0.5 | 0.75 | 1 | 1.5 | 2 | 2.5 |
| 1.5 mol/L Tris | 0.02 | 0.03 | 0.04 | 0.06 | 0.08 | 0.1 |
| (pH 6.8) | | | | | | |
| 10% Ammonium | 0.02 | 0.03 | 0.04 | 0.06 | 0.08 | 0.1 |
| persulfate | | | | | | |
| TEMED | 0.002 | 0.003 | 0.004 | 0.006 | 0.008 | 0.01 |

2. Formula of loading buffer: 50 mmol/L Tris-HCl pH 6.8, 0.05% w/v SAR, 0.1% w/v bromophenol blue, 10% v/v glycerin;

3. The formula of running buffer: 25 mmol/L Tris, 0.25 M/L glycine, 0.05% w/v SAR

4. Formula of protein Marker

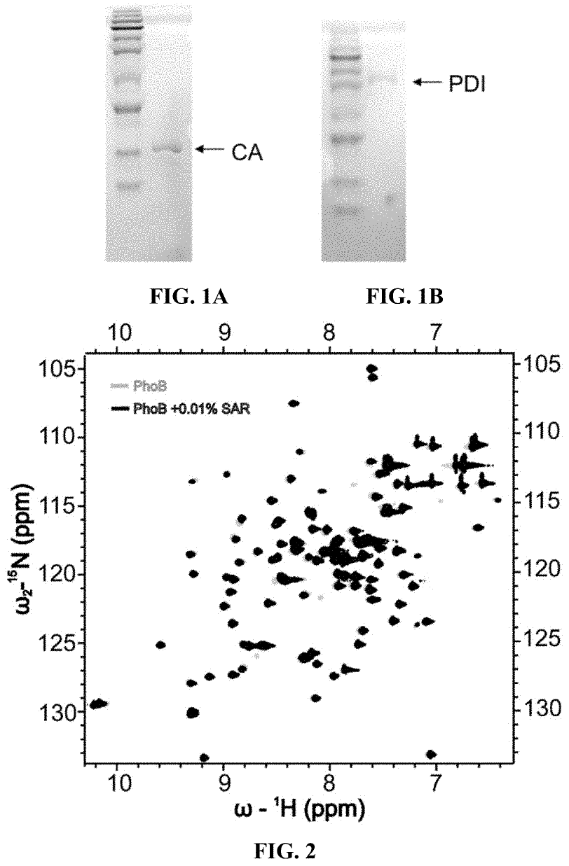

The analysis results were shown in FIGS. 1A-1B. The results showed that 05SAR-PAGE could be used to effectively determine the molecular mass of proteins.

### Example 2

Effect of SAR Concentration on Protein Structure

The ¹-¹⁵N HSQC spectrum of PhoB_{N}, the ¹-¹⁵N HSQC spectrum of PhoB_{N} added with 0.01% (w/v) SAR, and the ¹H-¹⁵N HSQC spectrum of PhoB_{N} added with 0.1% (w/v) SAR were obtained respectively, and then they were superimposed, to determine the chemical shift perturbation of amino acid residues.

The superimposition results of ¹-¹⁵N HSQC spectra of PhoB_{N}, and PhoB_{N} added with 0.01% (w/v) SAR were shown in FIG. 2. The gray signals represented the ¹H-¹⁵N HSQC spectrum of PhoB_{N}, and the black signals represented the ¹-¹⁵N HSQC spectrum of PhoB_{N} added with 0.01% (w/v) SAR. After 0.01% (w/v) SAR was added to 0.1 mmol/L PhoB_{N}, the chemical shifts of related amino acid residues in the ¹-¹⁵N HSQC spectra were almost not disturbed, and PhoB_{N} was still in a structured state, namely, 0.01% (w/v) SAR would not destroy the structure of PhoB_{N}.

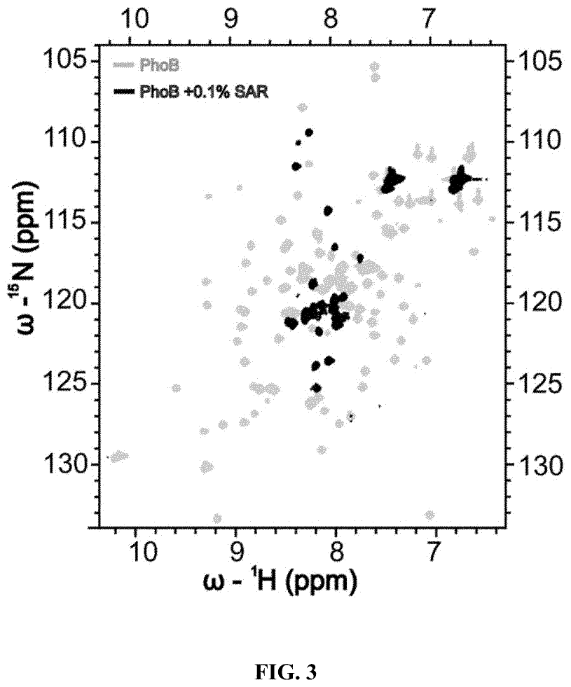

The superimposition results of ¹-¹⁵N HSQC spectra of PhoB_{N}, PhoB_{N} added with 0.1% (w/v) SAR were shown in FIG. 3. The gray signals represented the ¹-¹⁵N HSQC spectrum of PhoB_{N}, and the black signals represented the ¹-¹⁵N HSQC spectrum of PhoB_{N} added with 0.1% (w/v) SAR. After 0.1% (w/v) SAR was added to 0.1 mmol/L PhoB_{N}, the chemical shifts of amino acid residues in the ¹-¹⁵N HSQC spectra were greatly disturbed and PhoB_{N} became unstructured, namely, 0.1% (w/v) SAR would cause a large change in the structure of PhoB_{N}. Although SAR gel electrophoresis could still be used for protein molecular mass quantitative analysis, the accuracy of the analytic results of protein's natural conformation was low.

### Example 3

Effect of Loading Protein Concentration

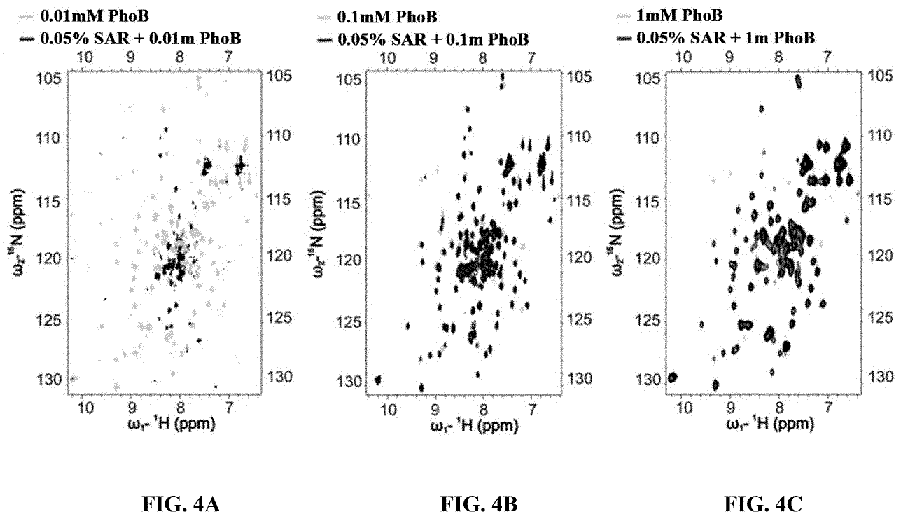

Samples with different concentrations of PhoB_{N} (0.01 mmol/L PhoB_{N}, 0.10 mmol/L PhoB_{N} and 1 mmol/L PhoB_{N}) were prepared as loading protein samples. The PhoB_{N} at different concentrations was titrated with 0.05% (w/v) SAR respectively, and the ¹-¹⁵N HSQC spectra of PhoB_{N} (FIGS. 4A-4C) were collected. As shown from the spectra results, after 0.05% (w/v) SAR was added to 0.01 mmol/L PhoB_{N}, the chemical shifts of the amino acids of PhoB_{N} were changed completely to an unstructured state (FIG. 4A); while after 0.05% (w/v) SAR was added to 0.10 mmol/L PhoB_{N} and 1 mmol/L PhoB_{N}, the entire structure of PhoB_{N} was not destroyed by SAR. According to the spectra results, as PhoB_{N} concentration increased, the number of amino acids with chemical shift changes in the ¹-¹⁵N HSQC spectra decreased accordingly, indicating that 0.10 mmol/L-1 mmol/L PhoB_{N} was stable in 0.05% (w/v) SAR.

In FIGS. 4A-4C, the gray signals represented the ¹-¹⁵N HSQC spectrum of PhoB_{N}, and the black signals represented the ¹-¹⁵N HSQC spectra of PhoB_{N} at different concentrations after adding SAR. Where, (FIG. 4A) the black signal represented the ¹H-¹⁵N HSQC spectrum of 0.01 mmol/L PhoB_{N} added with 0.05% (w/v) SAR; (FIG. 4B) the black signal represented the ¹-¹⁵N HSQC spectrum of 0.10 mmol/L PhoB_{N} added with 0.05% (w/v) SAR; (FIG. 4C) the black signal represented the ¹-¹⁵N HSQC spectrum of 0.01 mmol/L PhoB_{N} added with 0.05% (w/v) SAR.

As shown from FIGS. 4A-4C, 0.01 mmol/L protein was denatured by 0.05% SAR, i.e., the ratio of the molar concentration of the surfactant to the mass concentration of the loading protein was not higher than 11.56 mmol/g, otherwise the protein would bind to excessive SAR, leading to protein denaturation.

### Example 4

Comparison of 05SAR-PAGE Results of Loading Proteins at Different Mass Concentrations

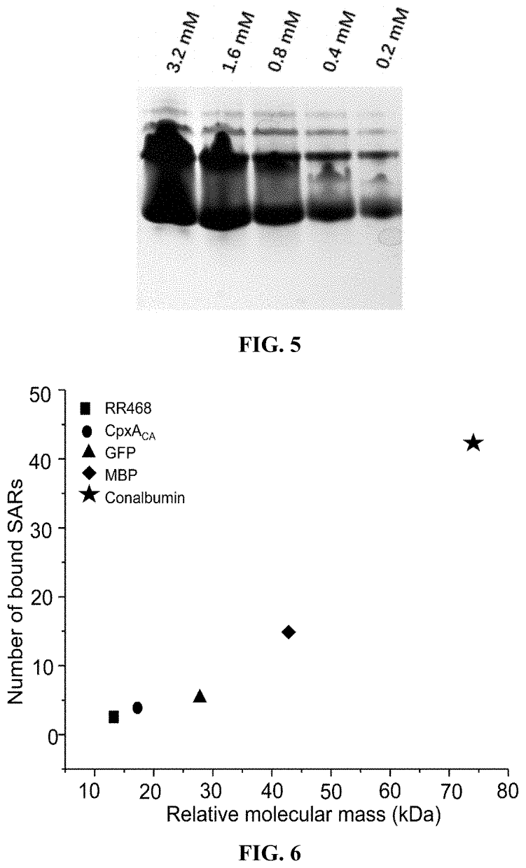

In order to explore whether a high concentration of protein would affect the quality of 05SAR-PAGE patterns, different concentrations of cytochrome c (Cyt c) samples (0.2 mmol/L, 0.4 mmol/L, 0.8 mmol/L, 1.6 mmol/L, and 3.2 mmol/L) were prepared, and conducted 05SAR-PAGE experiment according to the formula and steps of Example 1. The results were shown in FIG. 5.

The experimental results showed that, when the sample concentration gradually increased, tailing appeared for the protein samples, which affected the quality of the 05SAR-PAGE patterns. This indicated that, excessively high protein concentration would cause the appearance of obvious mid-tailing in the gel electrophoresis patterns. When the sample concentration of Cyt c was lower than 0.2 mmol/L, high quality of gel electrophoresis patterns could be obtained.

In 0.05% SAR, 3.2 mmol/L protein reduced the resolution of gel electrophoresis. Therefore, too high concentration of protein would affect the resolution of the gel, i.e., the ratio of the molar concentration of the surfactant to the mass concentration of the loading protein was not lower than 0.04 mmol/g.

### Example 5

Experiment on the Ratio of the Molar Concentration of the Surfactant to the Mass Concentration of the Loading Protein

By collecting the ¹H-NMR spectrum of SAR and titrating the known concentration of SAR with a known concentration of protein, the number of SAR bound to each protein was calculated according to the change in the integral area of SAR, as shown in FIG. 6. The relative molecular mass of the protein was plotted against the number of bound SAR, and it was found that, the larger the relative molecular mass of the protein, the more bound SAR, exhibiting a positive correlation and a good linear relationship, R²: 0.96. This good linear relationship showed that the amount of bound SAR was positively linearly correlated with the molecular mass of the protein during the process of 05SAR-PAGE.

As shown in FIG. 6, by calculation, the ratio of the molar concentration of SAR to the mass concentration of the loading protein was in the range of 1.66 mmol/g to 9.90 mmol/g (derived from the MSP protein), and there was a good linear relationship between the molecular weight of the protein and the amount of bound SAR. The linear relationship was good, R²=0.96.

In order to further explore the relationship between the protein migration rate and the protein molecular mass in 05SAR-PAGE, a processed protein Marker was made from the purchased protein Marker. After adsorbing the SDS with biobeads, 0.05% w/v SAR was added to the processed protein Marker. The proteins in the processed Marker were numbered 1 to 10 according to the molecular mass. As shown in the table below, the relative molecular masses of all proteins were counted and their log M values were calculated. In addition, a 05SAR-PAGE experiment was performed on the processed protein Marker. Assuming that the migration distance of bromophenol blue was d1 and the migration distance of the protein was d2, the migration rate Rm of various proteins was calculated according to the following formula.

Rm=d2/d1

According to the migration position of the protein electrophoresis, the migration distances of bromophenol blue and each protein in 05SAR-PAGE were measured with a caliper. The experiment was repeated three times, and the average migration rate dm of each protein was calculated. The statistical results were shown in Table 7.

TABLE 7
Molecular mass and migration rates of proteins in 05SAR-PAGE

| Protein | Molecular mass | log | Migration rate |
|---|---|---|---|
| No. | M (104 Da) | (M) | (Average (dm) of three measurements) |
| 1 | 18.00 | 1.26 | 0.02 |
| 2 | 14.00 | 1.15 | 0.03 |
| 3 | 10.00 | 1 | 0.04 |
| 4 | 7.50 | 0.88 | 0.06 |
| 5 | 6.00 | 0.78 | 0.09 |
| 6 | 4.50 | 0.65 | 0.12 |
| 7 | 3.50 | 0.54 | 0.2 |
| 8 | 2.50 | 0.4 | 0.29 |
| 9 | 1.50 | 0.18 | 0.43 |
| 10 | 1.00 | 0 | 0.53 |

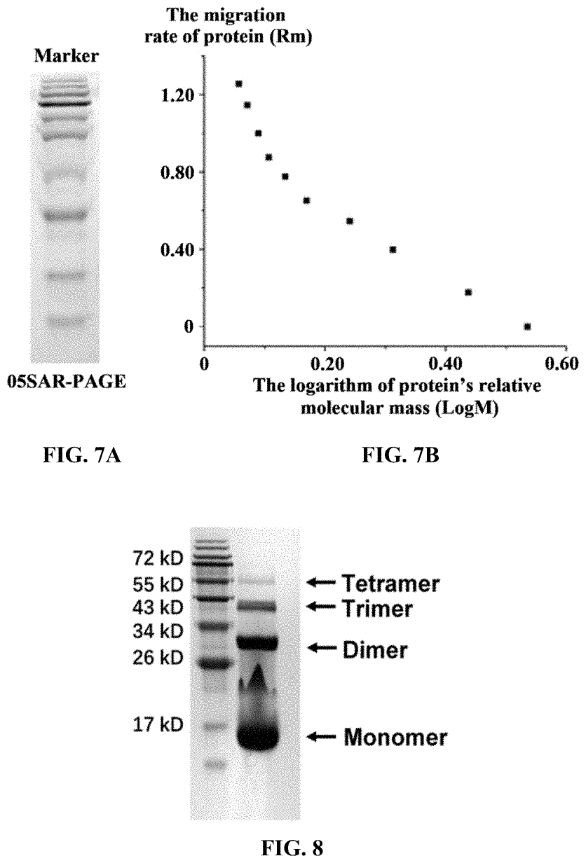

Using the logarithm of the relative molecular mass of each protein as the abscissa and the average migration rate of each protein molecule as the ordinate, a point plot was drawn, as shown in FIGS. 7A-7B.

According to the statistical experimental data, when the molecular mass was in the range of 10 kDa-180 kDa, the greater the relative molecular mass of the protein was, the faster its migration rate in 05SAR-PAGE was, which indicated that the migration rate of the protein was positively correlated to the logarithm of the relative molecular mass in 05SAR-PAGE. Thus, a universal protein Marker could be used for calibration of the molecular mass of the protein.

### Example 6

Separation and Determination of Polymerization States of a Protein Using 05SAR-PAGE

The gel electrophoresis system used was similar to that in Table 5 of Example 1, and the ratio of the molar concentration of the surfactant to the mass concentration of the loading protein was 0.04-11.56 mmol/g.

The Cyt c of yeast cells was used as a research object, and 0.5 mmol/L Cyt c sample and 05SAR-PAGE at a concentration of 15% were prepared for gel electrophoresis experiments, i.e., the ratio of the molar concentration of the surfactant to the mass concentration of the loading protein was 0.27 mmol/g. Through protein Marker comparison, the 05SAR-PAGE of Example 1 was used for electrophoresis. The results were shown in FIG. 8.

The results showed the bands of Cyt c monomer, dimer, trimer and tetramer (FIG. 8), indicating that 05SAR-PAGE could not only be used to detect the dimerization state of proteins, but also detect the oligomers and superpolymers of proteins. Under this condition, the oligomerization states and monomers of Cyt c could be clearly identified.

### Example 7

Protein Separation and Determination of Homo-Oligomeric State of Protein Using 05SAR-PAGE

The gel electrophoresis system used was similar to the gel electrophoresis system in Table 5 of Example 1, and the ratio of the molar concentration of the surfactant to the mass concentration of the loading protein was 0.04-11.56 mmol/g.

PhoB_{N} has an isoelectric point of 4.64, which is an acidic protein; PhoR_{CP} has an isoelectric point of 8.0, which is an alkaline protein. According to early literatures, both of them have a dimerization state; in addition, the interface is formed by weak interactions between molecules, so the two proteins were selected as the samples.

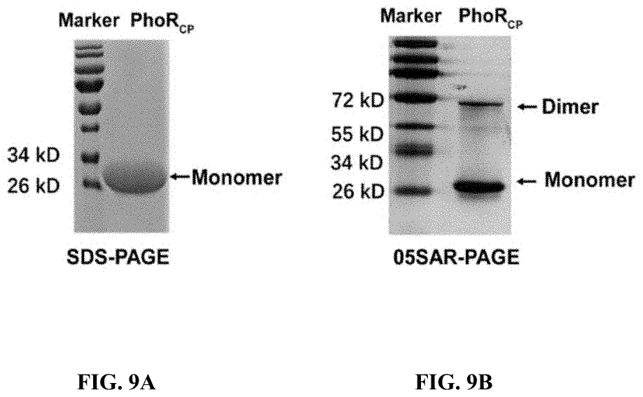

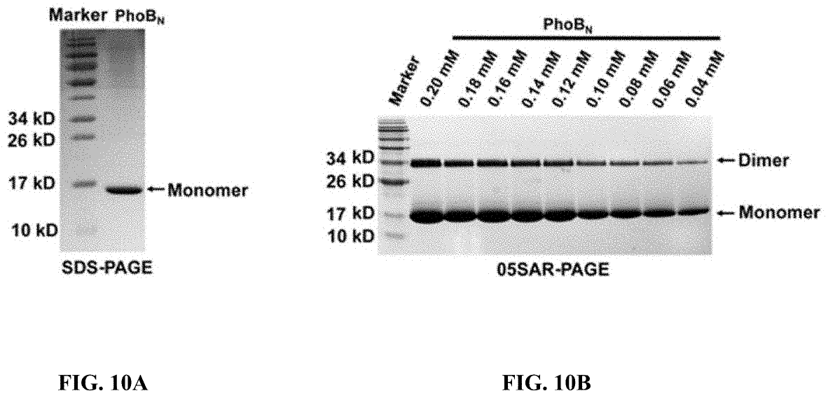

The PhoR_{CP} and PhoB_{N} samples were placed under the conditions of a refrigerator at 4° C. and a constant voltage of 90V for 05SAR-PAGE experiments. The results were shown in FIGS. 9A-9B and FIGS. 10A-10B.

For PhoR_{CP} protein, in SDS-PAGE, PhoR_{CP} was in a monomer state (FIG. 9A), indicating that the weak interaction between PhoR_{CP} dimers could be broken by SDS. Two bands appeared in the 05SAR-PAGE of PhoR_{CP}. Through the molecular mass analysis by the protein Marker, they were the monomer and dimer of PhoR_{CP}. The experimental results showed that, 05SAR-PAGE could be used to analyze the dimerization state of basic protein PhoR_{CP} in the solution. The concentration of PhoR_{CP} was 0.3 mmol/L, i.e., the ratio of the molar concentration of the surfactant to the mass concentration of the loading protein was 0.19 mmol/g, and the PhoR_{CP} protein bands in different aggregation states were clearly visible under this condition.

For PhoB_{N} protein, only PhoB_{N} monomer state (FIG. 10A) could be observed in SDS-PAGE, but the 05SAR-PAGE experiment clearly showed that PhoB_{N} existed in two bands at the same time. Through the molecular mass analysis of the two bands by the protein Marker, they were the monomer and dimer of PhoB_{N}. When the concentration of PhoB_{N} was as low as 0.04 mmol/L, the dimerization state of PhoB_{N} could still be observed in 05SAR-PAGE (FIG. 10B). This experimental phenomenon was consistent with previously reported experiments, indicating that 05SAR-PAGE was a milder gel than conventional SDS-PAGE and could be used to analyze the dimerization state of acidic protein PhoB_{N} in solution. The concentration of PhoB_{N} was 0.04-0.20 mmol/L, i.e., the ratio of the molar concentration of the surfactant to the mass concentration of the loading protein was 0.58-2.89 mmol/g, and the protein bands were clearly visible under this condition.

Note: 05SAR-PAGE overcame the shortcomings of conventional Native-PAGE, and could be calibrated with protein Marker. It could be used to separate and identify the dimerization state of acidic and basic proteins in one step.

### Example 8

Identification of the Aggregation States of Membrane Proteins

The gel electrophoresis system used was similar to that in Table 4 of Example 1, and the ratio of the molar concentration of the surfactant to the mass concentration of the loading protein was 0.04-11.56 mmol/g.

The solution environment of membrane proteins is complicated, so it is difficult to identify the aggregation states of membrane proteins. The loading buffer comprising SAR as a surfactant provided in this example could be used to dissolve membrane proteins well, maintain the multimeric states as far as possible, and could be charged to facilitate the electrophoresis.

The purified CpxA sample was dissolved in a buffer comprising 20 mmol/L Tris, 150 mmol/L NaCl, 1 mmol/L EDTA, 0.05% (w/v) SAR.

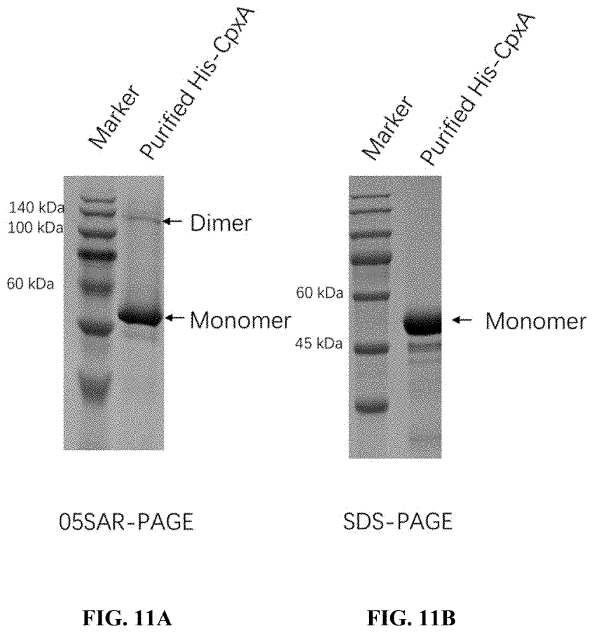

0.2 mmol/L CpxA samples were placed in a refrigerator at 4° C. and a constant voltage of 90V for SDS-PAGE and 05SAR-PAGE experiments. The results were shown in FIGS. 11A-11B.

As shown from the SDS-PAGE results on the right, the full-length CpxA was a monomer, but the 05SAR-PAGE results on the left indicated that the full-length CpxA had both monomer and dimer states, indicating that the full-length CpxA may have an oligomeric state in a solution. Due to the low expression level, large molecular mass, hydrophobicity property region, and complex dissolving buffer of a membrane protein, it is still difficult to detect the oligomerization state of membrane proteins. herein, 05SAR-PAGE was used to detect the oligomerization state of a membrane protein. According to the reports in the previous literatures, there was dimerization in intracellular full-length CpxA. This result was consistent with the results reported in the literatures. The concentration of CpxA was 0.20 mM, i.e., the ratio of the molar concentration of the surfactant to the mass concentration of the loading protein was 0.16 mmol/g, and the protein bands were clearly visible under this condition.

Note: 05SAR-PAGE was suitable for separating and identifying a dimerization state of membrane proteins.

### Example 9

Identification of Protein Modification States

The gel electrophoresis system used was similar to that in Table 5 of Example 1, and the ratio of the molar concentration of the surfactant to the mass concentration of the loading protein was 0.17-2.70 mmol/g.

Preparation of PhoB_{N} Phosphorylated Samples:

PAM (monoanhydride with phosphoric acid) is a commonly used small molecule phosphate donor that can mimic phosphorylation of PhoB_{N}. 4.4 mg of PhoB_{N} protein sample was dissolved in 500 μL of reaction buffer (20 mmol/L Tris, 50 mmol/L NaCl and 20 mmol/L MgCl₂), and adjusted to pH 8, then 3 mmol/L PAM was added, and placed at 25° C. water bath for the phosphorylation reaction. Samples were taken once at an interval of 1.5 min (namely, 0, 1.5, 3, 4.5, 6, 7.5, 9, 10.5, 12 min), and each sample was quickly frozen in liquid nitrogen to terminate the reaction. In 0 min, no PAM was added to PhoB_{N}; at the same time, as the control group of this experiment, the non-phosphorylated PhoB_{N} samples without adding PAM under the same condition were placed at 25° C. water bath. Samples were taken once at an interval of 1.5 min (namely, 0, 1.5, 3, 4.5, 6, 7.5, 9, 10.5, 12 min), and each sample was quickly frozen in liquid nitrogen to terminate the reaction. The concentration of PhoB_{N} was 0.30 mmol/L, i.e., the ratio of the molar concentration of the surfactant to the mass concentration of the loading protein was 0.39 mmol/g.

Preparation of Methylated Samples of Cyt c:

The concentration of Cyt c was 0.05 mmol/L, i.e., the ratio of the molar concentration of the surfactant to the mass concentration of the loading protein was 2.7 mmol/g. The experimental results at this concentration were shown in Figure b below. The Cyt c methylated and unmethylated samples could be clearly identified. When the concentration of Cyt c reached 0.8 mmol/L, i.e., the ratio of the molar concentration of the surfactant to the mass concentration of the loading protein was 0.17 mmol/g, the Cyt c methylated and unmethylated samples could still be identified.

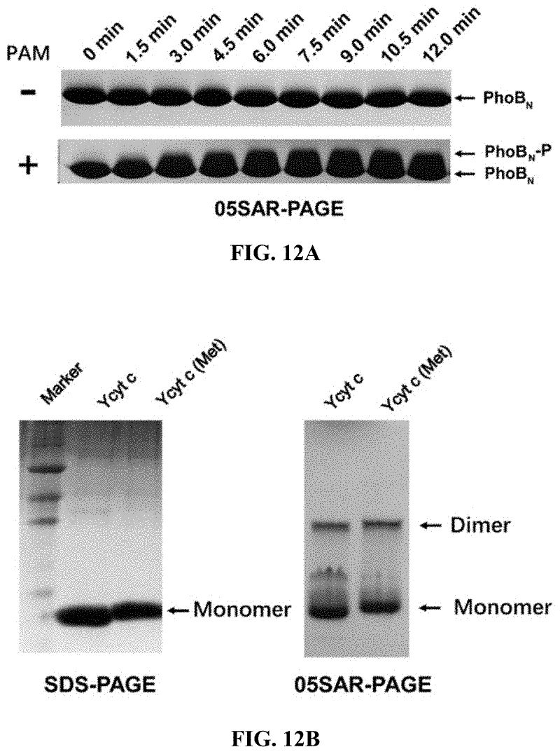

A 05SAR-PAGE was performed on 4° C. ice at a voltage of 90V. The duration of electrophoresis was set to 5 hours. The results were shown in FIGS. 12A-12B.

In FIG. 12A, the phosphorylation degree of PhoB_{N} could be identified and a wider band was formed. In FIG. 12B, the monomer and dimer of Cyt c were marked with arrows to indicate the chemical modification states of protein. The targeting modification of protein is of great significance to the function of proteins in organisms. As 0.05% w/v SAR has a mild effect on protein structure, 05SAR-PAGE can be used to study the protein modification. In the experiment, the methylation modification of Cyt c and the phosphorylation modification of PhoB_{N} were observed by 05SAR-PAGE. In addition, compared with the conventional SDS-PAGE, the methylation modification of Cyt c in the dimeric state was observed.

### Example 10

Identification of Protein Polymerization State Combined with Western Blotting

The gel electrophoresis system used in the 05SAR-PAGE experiment was similar to that in Table 4 of Example 1. The ratio of the molar concentration of the surfactant to the mass concentration of the loading protein was 0.04-5.31 mmol/g.

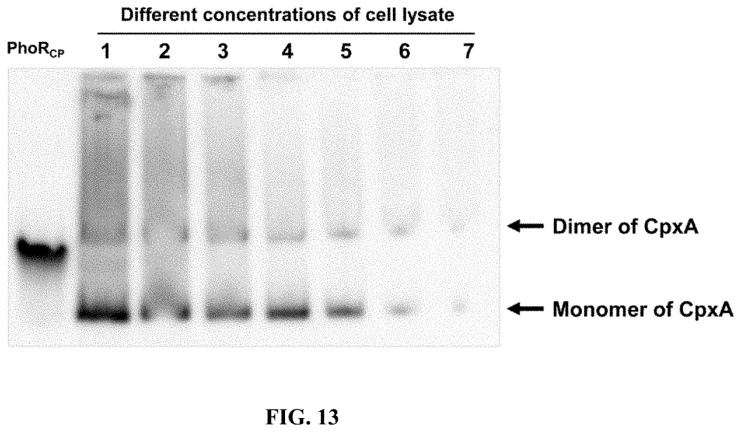

When 05SAR-PAGE was used for Western Blotting to observe the polymerization state of intracellular proteins and the polymerization state of membrane proteins, the suitable ratio of the molar concentration of SAR to the mass concentration of the loading protein was 0.04-5.31 mmol/g, since the staining method of Western Blotting was more sensitive than the PAGE. As shown in FIG. 13, the amount of PhoR_{CP} was 12 μg. Lanes 1-6 were E. coli cell lysates of different concentrations. According to the gray scale calculation, when the minimum concentration of the membrane protein CpxA was 3.24 μg (lane 5), the dimer band of CpxA could still be observed and the calculated ratio was 5.31 mmol/g. If it was higher than 5.31 mmol/g, the gray scale of the gel was low, the polymerization state of CpxA would not be clearly observed. If it was lower than 0.04 mmol/g, the gel tailing phenomenon would appear due to high concentration of protein, which would affect the resolution of gel electrophoresis.

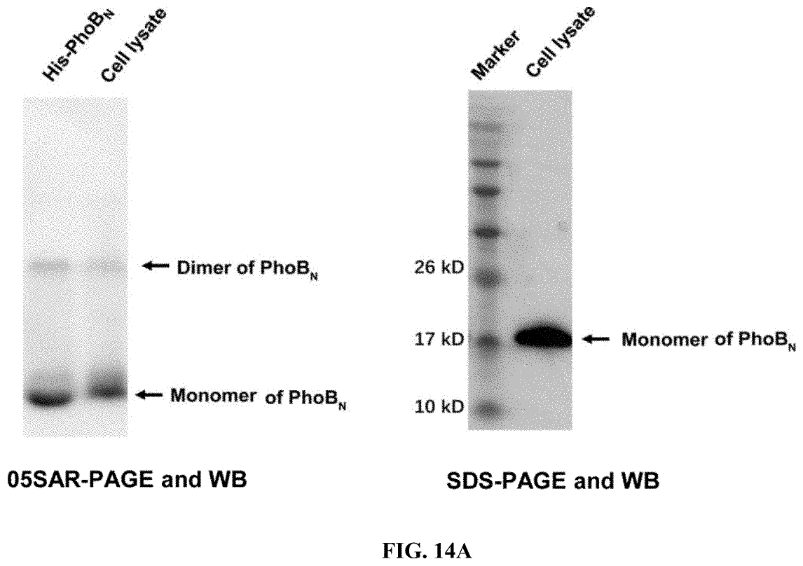

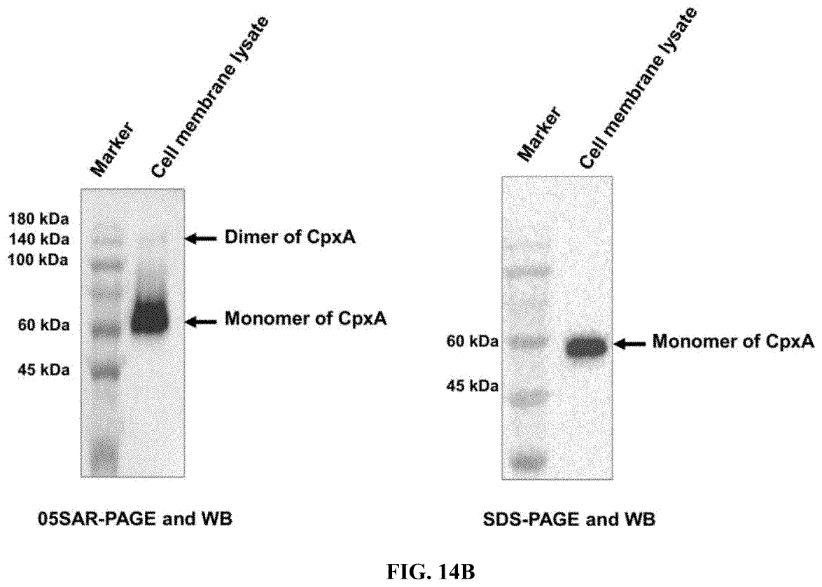

Electrophoresis conditions and procedures of Western Blotting: Resuspend the bacterial cells in 50 mmol/L Tris, 100 mmol/L NaCl, pH 8.0. Sonicate the bacteria on ice for 10 minutes to obtain the cell lysate. Perform SDS-PAGE and 05SAR-PAGE at 4° C. and 90V, and then transfer to a nitrocellulose (NC) membrane by a semi-dry transfer device at 15 V for 30 minutes. Incubate the NC membrane with 5% (w/v) skimmed milk in TBST (20 mmol/L Tris-HCl, 500 mmol/L NaCl, 0.05% Tween-20, pH 8.0) for 1.5 h, and then incubate with the mouse anti-His monoclonal antibody (1:3000). The secondary antibody was goat anti-mouse IgG-HRP (1:3000). After washing with TBST for five times, add a clear Western ECL substrate solution, and scan the gels using a Gel scanner. The results were shown in FIGS. 14A-14B.

For proteins with high expression levels in cells, 05SAR-PAGE could be combined with immunohybridization experiments to detect the aggregation states of proteins in cells.

### Example 11

05SAR-PAGE and SDS-PAGE were used for two-dimensional electrophoresis, combined with Western blot and mass spectrometry, the protein complexes in cell lysates were identified.

The gel electrophoresis system used in the 05SAR-PAGE experiment was similar to the gel electrophoresis system in Table 3 or 4 in Example 1, and the ratio of the molar concentration of the surfactant to the mass concentration of the loading protein was 0.04-5.31 mmol/g.

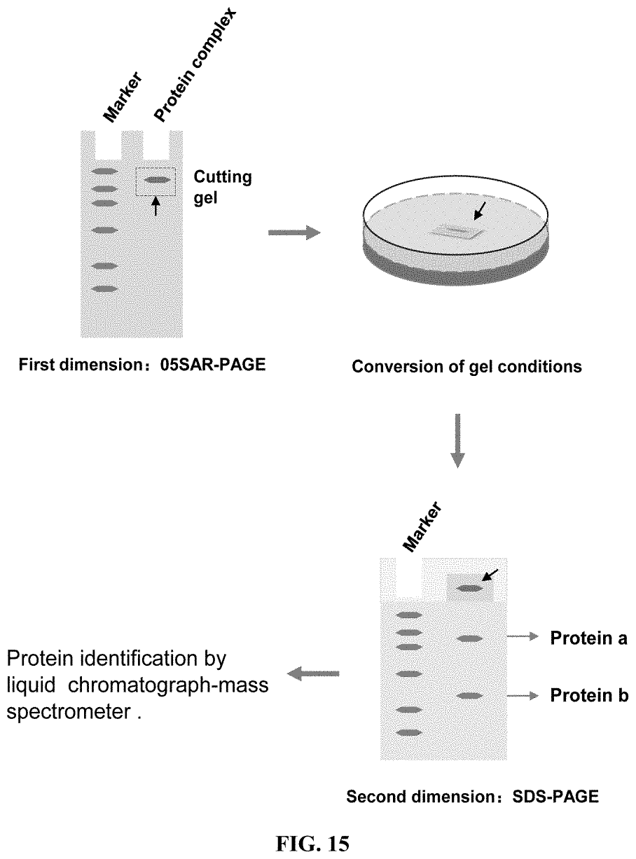

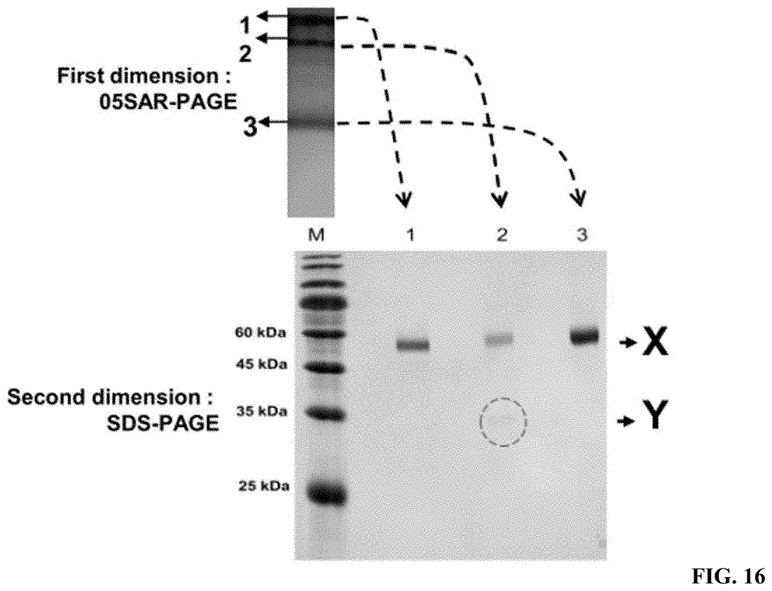

05SAR-PAGE was combined with SDS-PAGE to form two-dimensional electrophoresis, as shown in FIG. 15. CpxA was overexpressed in E. coli, and the whole membrane lysate of E. coli was collected. 05SAR-PAGE and Western blotting were used to separate and identify the bands of CpxA in the entire membrane lysate, as shown in FIG. 16. The monomer, dimer and complex of CpxA were successfully separated by two-dimensional 05SAR/SDS-PAGE, as shown in FIG. 16. X was identified as membrane protein CpxA and Y as membrane protein OmpA by liquid chromatography-mass spectrometry.

Experiments showed that two-dimensional 05SAR/SDS-PAGE can identify membrane protein complexes in the membrane lysate, and is expected to be used for complex protein system and proteomics research, to obtain and analyze protein interaction data.

It will be obvious to those skilled in the art that changes and modifications may be made, and therefore, the aim in the appended claims is to cover all such changes and modifications.

The invention claimed is:

1. A gel for protein electrophoresis, comprising separating gel and a stacking gel disposed on the separating gel; wherein the separating gel is a polyacrylamide gel comprising a surfactant, and is alkaline; the surfactant of the separating gel comprises 0.025-0.1% (m/v) of sodium lauroyl sarcosinate; a ratio of a molar concentration of the surfactant to a mass concentration of a loading protein is between 0.04 mmol/g and 11.56 mmol/g.

2. The gel of claim 1, wherein the ratio of the molar concentration of the surfactant to the mass concentration of the loading protein is between 1.16 mmol/g and 9.90 mmol/g.

3. The gel of claim 1, wherein the stacking gel comprises polyacrylamide with a mass volume fraction of 3.5-5%, and the surfactant of the stacking gel comprises 0.025-0.1% (m/v) of sodium lauroyl sarcosinate.

4. A buffer for protein electrophoresis, the buffer comprising a surfactant; the surfactant comprising 0.025-0.1% (m/v) of sodium lauroyl sarcosinate, and a ratio of a molar concentration of the surfactant to a mass concentration of a loading protein is between 0.04 mmol/g and 11.56 mmol/g.

5. The buffer of claim 4, being a loading buffer, a running buffer, or a buffer for dissolving a protein Marker.

6. The buffer of claim 5, wherein the buffer is the loading buffer, and a mass concentration of the loading protein is not more than 40.32 mg/mL.

7. The buffer of claim 6, wherein a mass concentration of the loading protein for the buffer for protein electrophoresis is 0.59-2.94 mg/mL.

8. A Marker for protein electrophoresis, comprising 4 to 20 kinds of stabilized monomer proteins with a molecular mass distribution range of 5 kDa to 100 MDa, and a total protein concentration being 0.005 mmol/L to 0.5 mmol/L; wherein the stabilized monomer proteins refer to proteins used as a Marker, and no polymer is formed in the proteins themselves or between the proteins; a dissolution buffer of the stabilized monomer proteins comprises sodium lauroyl sarcosinate with the same concentration as a stacking gel for protein electrophoresis.

9. The Marker of claim 8, comprising 10 kinds of stabilized monomer proteins.

10. A method for separation and determination of polymerization states of proteins, observation of modification states of a proteins, observation of a protein complex, analysis of protein purity, determination of protein molecular mass, protein identification by Western Blotting, detection of an aggregation states of intracellular and extracellular membrane proteins, or identification of protein complexes separated by two-dimensional 05SAR/SDS-PAGE, the method comprising applying the gel for protein electrophoresis of claim 1.

11. The method of claim 10, wherein
when the gel for protein electrophoresis is applied to separation and determination of polymerization states of protein, observation of a protein complex, analysis of protein purity, determination of protein molecular mass, or detection of an aggregation states of intracellular and extracellular membrane proteins, the ratio of a molar concentration of the surfactant to the mass concentration of the loading protein is between 0.04 mmol/g and 11.56 mmol/g;
when the gel for protein electrophoresis is applied to observation of modification states of protein, the ratio of a molar concentration of the surfactant to the mass concentration of the loading protein is between 0.17 mmol/g and 2.31 mmol/g; and
when the gel for protein electrophoresis is applied to protein identification by Western Blotting, or identification of protein complexes separated by two-dimensional 05SAR/SDS-PAGE, the ratio of a molar concentration of the surfactant to the mass concentration of the loading protein is between 0.04 mmol/g and 5.31 mmol/g.

12. The method of claim 11, wherein identification of protein complexes separated by two-dimensional 05SAR/SDS-PAGE comprises:
carrying out gel electrophoresis on a cell lysate by 05SAR-PAGE; cutting the gel after electrophoresis and recovering proteins in each lane according to protein bands, thereby yielding protein series; and
carrying out gel electrophoresis on the protein series with SDS-PAGE, respectively, and isolating and obtaining interacting protein data.

13. A kit for protein gel electrophoresis, comprising a stock solution of the stacking gel and a stock solution of the separating gel of the gel for protein electrophoresis of claim 1.

14. The kit of claim 13, further comprising a Marker for protein electrophoresis; the Marker for protein electrophoresis comprising 4 to 20 kinds of stabilized monomer proteins with a molecular mass distribution range of 5 kDa to 100 MDa, and a total protein concentration being 0.005 mmol/L to 0.5 mmol/L; wherein the stabilized monomer proteins refer to proteins used as a Marker, and no polymer is formed in the proteins themselves or between the proteins; a dissolution buffer of the stabilized monomer proteins comprises sodium lauroyl sarcosinate with the same concentration as a stacking gel for protein electrophoresis.

15. The kit of claim 14, wherein the stock solution of the separating gel comprises a buffer comprising sodium lauroyl sarcosinate and acrylamide at a formulation concentration, ammonium persulfate solution, and a tetramethylethylenediamine (TEMED) solution.
